(19) United States
(12) Patent Application Publication
(10) Pub. No.: US 2009/0221615 A1
(43) Pub. Date: Sep. 3, 2009

(54) COMBINATION ANTI-CANCER AGENTS

(76) Inventors: Guru Reddy, Irvine, CA (US); Luigi Lenaz, Newton, PA (US)

Correspondence Address: K&L Gates LLP, 1900 MAIN STREET, SUITE 600, IRVINE, CA, 92614-7319, US

(21) Appl. No.: 12/389,318

(22) Filed: Feb. 19, 2009

Related U.S. Application Data
(60) Provisional application No. 61/032,831, filed on Feb. 29, 2008.

(51) Int. Cl. A61K 31/522; A61K 31/382; A61P 35/00

(52) U.S. Cl. 514/265.1; 514/437

(57) ABSTRACT

The present disclosure relates to methods of treating cancer in mammals by administration of Lucanthone and at least one anti-metabolite. Pharmaceutical compositions and kits comprising Lucanthone and at least one anti-metabolite also are disclosed.

### CROSS REFERENCE TO RELATED APPLICATIONS

This application claims the benefit of U.S. provisional patent application No. 61/032,831 filed Feb. 29, 2008, the entire disclosure of which is incorporated herein by reference.

### FIELD OF THE INVENTION

The present disclosure relates to methods of treating cancer in mammals by administration of Lucanthone and at least one anti-metabolite. Pharmaceutical compositions and kits comprising Lucanthone and at least one anti-metabolite also are disclosed.

### BACKGROUND OF THE INVENTION

Lucanthone is a thioxanthione. Lucanthone was discovered and developed in the 1930's and 1940's to treat schistosomiasis in humans. Lucanthone was used to treat schistosomiasis patients worldwide in the 1950's and 1960's with an excellent safety record.

Lucanthone also has antitumor activity. Hirschberg et al. (J. Natl. Cancer Inst. (1959) 22, 567-579) were the first to study the antitumor activity of Lucanthone in vitro. Subsequently, Lucanthone was tested at the National Cancer Institute for in vitro anti-tumor activity against a panel of 60 cell lines. The results of the study showed that Lucanthone had in vitro anti-tumor activity against all the cell lines tested at μM concentrations. The ranges of GI50 (concentration required for 50% growth inhibition), TGI (concentration required for 100% growth inhibition), and LC50 (concentration required for 50% decrease in cellular protein) were relatively narrow which indicate that the sensitivity of the cell lines to Lucanthone did not vary widely.

Lucanthone, at a concentration of 3 μg/mL, has been shown also to enhance X-ray damage in HeLa cells, probably by inhibiting postradiation repair process. The radiation sensitizing effect of Lucanthone was dependent on exposure time and was reversible. Lucanthone's ability to inhibit topoisomerase II and apurinic/apyrimidinic (AP) endonuclease that lead to inhibition of DNA repair probably accounts for the anti-tumor activity and radiation sensitizer activity of Lucanthone.

Radiation sensitizing effect of Lucanthone in CHO cells was studied by Leeper et al. (Int. J. Radiat. Oncol. Biol. Phys. 1978 March-April; 4 (3-4): 219-27), who showed that Lucanthone, at concentrations of 5 μg/mL or higher, reduced the capacity of CHO cells to accumulate and repair sublethal radiation damage in a time dependent manner, whether the drug is present before or after irradiation. The radiation sensitizing effect of Lucanthone was found to be reversible after removal of the drug. Similar observations were made by Durand et al. (Int. J. Radiat. Oncol. Biol. Phys. 1980 November; 6(11):1525-30), who studied the effect of Lucanthone in Chinese hamster V-79 cells. Lucanthone, at a concentration of 4 μM, was shown to enhance the cytotoxic activity of the alkylating agent temozolamide against MDA-MB231 breast cancer cells by two fold.

Antitumor activity of Lucanthone against a variety of tumors was studied by a number of investigators using mouse, rat and hamster models. In these studies, reviewed by Hirschberg (Antibiotics. New York: Springer Verlag; (1974) 3, 274-303), Lucanthone was shown to inhibit the growth of about half of the tumors tested without any discernible pattern of response. Lucanthone was also tested at the National Cancer Institute (NCI) against a number of tumors in mouse models. In these studies, Lucanthone was administered either subcutaneously or intraperitonially at doses up to 600 mg/kg following a variety of schedules. Mean tumor weight or median survival time, measured as percent of control, was used as an endpoint. In these studies, Lucanthone showed antitumor activity against approximately 30% of the tumors tested.

Lucanthone is an orally available small molecule inhibitor of apurinic/apyrimidinic (AP) endonuclease. Inhibition of AP endonuclease leads to accumulation of abasic sites in DNA that are converted to lethal double-strand breaks leading to sensitization of tumor cells to alkylating agents and to radiation. Because anti-metabolite agents, such as Pemetrexed, are also known to produce abasic sites in DNA, there exists a need for a combination therapy for cancer which utilizes both Lucanthone and anti-metabolite.

### SUMMARY OF THE INVENTION

The present disclosure relates to methods of treating cancer in mammals by concurrent or sequential administration of Lucanthone and at least one anti-metabolite. Pharmaceutical compositions and kits comprising Lucanthone and at least one anti-metabolite are also disclosed.

In one embodiment, the present disclosure relates to a method of treating cancer comprising administering to a mammal in need thereof a therapeutically effective amount of Lucanthone and a therapeutically effective amount of at least one anti-metabolite. Alternatively, Lucanthone and at least one anti-metabolite can be administered concurrently or sequentially. Alternatively, the administration of Lucanthone and at least one anti-metabolite can be within about three hours of each other. Alternatively, the administration of Lucanthone and at least one anti-metabolite can also be within about two hours of each other. Alternatively, the administration of Lucanthone and at least one anti-metabolite can also be within about one hour of each other. Alternatively, the cancer treated can be lung cancer. Alternatively, the lung cancer can be non-small lung carcinoma. Alternatively, the therapeutically effective amount of Lucanthone and the therapeutically effective amount of at least one anti-metabolite are administered in a single daily dose or divided into more than one daily dose. Alternatively, for all of the administrations of the Lucanthone and at least one anti-metabolite according to the present disclosure, the administration can be twice daily.

Alternatively, Lucanthone and at least one anti-metabolite according to the present treatment are administered orally. Alternatively, Lucanthone and the at least one anti-metabolite are administered parenterally. Alternatively, Lucanthone and the at least one anti-metabolite are administered in the form of a capsule or tablet. Alternatively, Lucanthone and the at least one anti-metabolite are administered for one or more cycles. Alternatively, one cycle comprises seven times once every four days.

In another embodiment, the present method of treating cancer further comprises administering radiation therapy, hormonal therapy or immunotherapy.

The present disclosure also relates to a pharmaceutical composition comprising therapeutically effective amounts of Lucanthone and at least one anti-metabolite, and a pharmaceutically acceptable carrier.

The present disclosure also relates to a pharmaceutical kit comprising: a pharmaceutical composition comprising a therapeutically effective amount of Lucanthone and a pharmaceutically acceptable carrier, and a pharmaceutical composition comprising a therapeutically effective amount of at least one anti-metabolite and a pharmaceutically acceptable carrier.

### BRIEF DESCRIPTION OF THE DRAWINGS

FIG. 1 is a graph showing the antitumor activity of Lucanthone and Pemetrexed as single agents and in combination.

### DETAILED DESCRIPTION OF THE INVENTION

Lucanthone is a chemotherapeutic or radiosensitizing intercalating agent. In the present disclosure, the term Lucanthone is taken to include Lucanthone proper, as 1-diethylaminoethylamino-4-methyl-10-thiaxanthenone, together with physiologically tolerated derivatives, analogs, and salts thereof. Such physiologically tolerated derivatives, analogs, and salts include, but are not limited to, hycanthone, indazole analogues of Lucanthone, and other analogs such as those disclosed in Thomas Corbett et al., Antitumor Activity of N-[[1-[[2-(diethylamino)ethyl]amino]-9-oxo-9H-thiaxanthen-4-yl]methyl]meth anesulfonamide (WIN33377) and analogues, Exp. Opin. Invest. Drugs 3:1281-1292 (1994); and Mark P. Wentland et al., Anti-solid Tumor Efficacy and Preparation of N-[[1-[[2-(diethylamino)ethyl]amino]-9-oxo-9H-thiaxanthen-4-yl]methyl]meth anesulfonamide (WIN33377) and Related Derivatives, Bioorg. & Med. Chem. Lett. 4:609-614 (1994).

Lucanthone has been used as a treatment for schistomiasis. Lucanthone has been known to have an cytotoxic or cytostatic effect on growing cells. The enhanced joint lethal action of Lucanthone and ionizing radiation in cells may be accounted for by the production of DNA double strand breaks (DSB) in cleavable complexes because of Lucanthone's inhibition of topoisomerase II, combined with the DSB induced by radiation alone. R. Bases, DNA Intercalating Agents as Adjuvants in Radiation Therapy, Int J Radiat Oncol Biol Phys 4:345-346 (1978) (editorial); R. E. Bases et al., Topoisomerase Inhibition by Lucanthone, an Adjuvant in Radiation Therapy, Int J Radiat Oncol Biol Phys 37:1133-1137 (1997).

Topoisomerase II may also be implicated in the mechanism of radiation induced DSB by an additional mechanism. When DNA bases are damaged by ionizing radiation, they are first removed by the cells' base excision repair enzymes, which first remove the damaged bases (by a glycosylase) and leave abasic sites. Removal of abasic sites is achieved in the second step, performed by endonucleases that cause strand scission and leave 3′ OH groups, which are required acceptors in DNA repair synthesis. Subsequent steps include removal of 5′ phosphate groups at the sites of excised bases, followed by gap filling by DNA polymerase beta, which inserts appropriate replacement nucleotides. DNA ligase completes repair by sealing in the replacement nucleotides.

An advantage of Lucanthone is that DNA replication requires topoisomerase II activity, thereby creating selective toxicity for cycling cells, such as cancerous cells. Normal cells, most of which do not cycle, would be therefore less sensitive to Lucanthone-based therapy, and would be less likely to be non-selectively damaged. Furthermore, Lucanthone's effects on bone marrow and the gut are moderate and reasonably quickly reversible.

An anti-metabolite as used herein is a chemical with a similar structure to a substance, such as a metabolite required for normal biochemical reactions, yet different enough to interfere with the normal functions of cells, including cell division. Anti-metabolites may interfere with DNA production and therefore cell division and the growth of tumors. Because cancer cells spend more time dividing than other cells, inhibiting cell division harms tumor cells more than other cells. Anti-metabolites may masquerade as purine (azathioprine, mercaptopurine) or pyrimidine which become the building blocks of DNA. They prevent these substances from becoming incorporated into DNA during the S phase (of the cell cycle), stopping normal development and division. They also affect RNA synthesis. However, because thymidine is used in DNA but not in RNA (where uracil is used instead), inhibition of thymidine synthesis via thymidylate synthase selectively inhibits DNA synthesis over RNA synthesis

One example of anti-metabolite is folate anti-metabolite. Another example of an anti-metabolite is Pemetrexed. Pemetrexed (sold or market under the brand name Alimta®) refers to Pemetrexed proper taken together with physiologically tolerated derivatives, analogs, and salts thereof. Its chemical name is N-[4-[2-(2-amino-4,7-dihydro-4-oxo-1H-pyrrolo[2,3-d]pyrimidin-5-yl)ethyl]-benzoyl]-L-glutamic acid. Pemetrexed can have an inhibitory effect on one or more enzymes which utilize folic acid, and in particular metabolic derivatives of folic acid, as a substrate. The compounds appear to be particularly active as inhibitors of thymidylate synthetase, which catalyzes the methylation of deoxyuridylic acid to deoxythymidylic acid. Pemetrexed thus can be used, alone or in combination, to inhibit the growth of neoplasms which otherwise depend upon the inhibited enzyme.

The inventors have unexpectedly discovered that Lucanthone and at least one anti-metabolite may be advantageously administered concurrently or sequentially in the treatment of cancers, wherein they may be administered in amounts effective to cause the arrest or regression of the cancer in the host.

The present disclosure relates to methods of treating cancer in mammals by concurrent or sequential administration of Lucanthone and at least one anti-metabolite. Pharmaceutical compositions and kits comprising Lucanthone and at least one anti-metabolite are also disclosed.

In one embodiment, the present disclosure relates to a method of treating cancer comprising administering to a mammal in need thereof a therapeutically effective amount of Lucanthone and a therapeutically effective amount of at least one anti-metabolite. “Therapeutically effective amount” refers to an amount of a compound that can be therapeutically effective to inhibit, prevent or treat the symptoms of a particular disease, disorder or side effect, for example, to cause arrest or regression of cancer.

Alternatively, the Lucanthone and at least one anti-metabolite can be administered concurrently or sequentially. This means that each component can be administered at the same time (concurrently) or sequentially in any order at different points in time. Also, concurrently as used may mean that the Lucanthone and at least one anti-metabolite may be taken together at the same time as part one pharmaceutical composition or together at the same time but in separate pharmaceutical compositions. Thus, each component can be administered separately but sufficiently closely in time so as to provide the desired therapeutic effect. Alternatively, the administration of Lucanthone and at least one anti-metabolite can be within about three hours of each other. Alternatively, the administration of Lucanthone and at least one anti-metabolite can be within about two hours of each other. Alternatively, the administration of Lucanthone and Pemetrexed can be within about one hour of each other.

Not all combinations of Lucanthone and anti-cancer agents will show the anti-tumor effect achieved with the present invention. Lucanthone and at least one anti-metabolite herein appear to work together to achieve results not possible with other combinations of known anti-cancer agents.

A broad range of cancers may be treated using the present invention. These cancers comprise both primary and metastatic cancers. As an example, the cancer treated can be lung cancer. Alternatively, the lung cancer can be non-small lung carcinoma.

The specific dose of Lucanthone and at least one anti-metabolite that are administered to obtain therapeutic or abrogatory effects will, of course, be determined by the particular circumstances surrounding the case, including, for example, the compound administered, the route of administration, the condition being treated and the individual host or patient being treated. Alternatively, the therapeutically effective amount of Lucanthone and the therapeutically effective amount of at least one anti-metabolite are administered in a single daily dose or divided into more than one daily dose. Alternatively, for all of the administrations of the Lucanthone and at least one anti-metabolite according to the present disclosure, the administration can be twice daily.

Lucanthone and Pemetrexed can be administered for one or more cycles. In one embodiment, one cycle comprises seven times once every four days.

In another embodiment, the present method of treating cancer further comprises administering radiation therapy, hormonal therapy or immunotherapy.

The present disclosure also relates to a pharmaceutical composition comprising therapeutically effective amounts of Lucanthone and Pemetrexed, and a pharmaceutically acceptable carrier.

The present disclosure also relates to a pharmaceutical kit comprising a pharmaceutical composition comprising a therapeutically effective amount of Lucanthone and a pharmaceutically acceptable carrier, and a pharmaceutical composition comprising a therapeutically effective amount of Pemetrexed and a pharmaceutically acceptable carrier.

The pharmaceutical compositions of the present disclosure include Lucanthone and Pemetrexed formulated into compositions together with one or more non-toxic physiologically acceptable carriers, adjuvants or vehicles which are collectively referred to herein as carriers. The pharmaceutical kits of the present disclosure comprise a pharmaceutical composition comprising Lucanthone and another pharmaceutical composition comprising Pemetrexed.

The pharmaceutical compositions and kits can be administered to humans and animals either orally, rectally, parenterally (intravenously, intramuscularly or subcutaneously), intracisternally, intravaginally, intraperitoneally, locally (powders, ointments or drops), or as a buccal or nasal spray. Either or both Lucanthone and anti-metabolite may be administered in a variety of routes, including orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In another embodiment, Lucanthone and at least one anti-metabolite are administered orally. Alternatively, Lucanthone and at least one anti-metabolite are administered parenterally. Alternatively, Lucanthone and at least one anti-metabolite are administered in the form of a capsule or tablet.

“Pharmaceutically acceptable” refers to those compounds, materials, compositions, and/or dosage forms which are, within the scope of sound medical judgment, suitable for contact with the tissues of human beings and animals without excessive toxicity, irritation, allergic response, or other problem complications commensurate with a reasonable benefit/risk ratio.

Compositions suitable for parenteral injection may comprise physiologically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethyleneglycol (PEG), glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

These compositions may also contain adjuvants such as preserving, wetting, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride and the like. Prolonged absorption of the injectable pharmaceutical form can be brought about by the use of agents delaying absorption, for example, aluminum monostearate and gelatin.

If desired, and for more effective distribution, the compounds can be incorporated into slow release or targeted delivery systems such as polymer matrices, liposomes, and microspheres. They may be sterilized, for example, by filtration through a bacteria-retaining filter, or by incorporating sterilizing agents in the form of sterile solid compositions which can be dissolved in sterile water, or some other sterile injectable medium immediately before use.

Solid dosage forms for oral administration include capsules, tablets, pills, powders and granules. In such solid dosage forms, the active compound may be admixed with at least one inert customary excipient (or carrier) such as sodium citrate or dicalcium phosphate or (a) fillers or extenders, as for example, starches, lactose, sucrose, glucose, mannitol and silicic acid, (b) binders, as for example, carboxymethylcellulose, alignates, gelatin, polyvinylpyrrolidone, sucrose and acacia, (c) humectants, as for example, glycerol, (d) disintegrating agents, as for example, agar-agar, calcium carbonate, potato or tapioca starch, alginic acid, certain complex silicates and sodium carbonate, (e) solution retarders, as for example, paraffin, (f) absorption accelerators, as for example, quaternary ammonium compounds, (g) wetting agents, as for example, cetyl alcohol and glycerol monostearate, (h) adsorbents, as for example, kaolin and bentonite, and (i) lubricants, as for example, talc, calcium stearate, magnesium stearate, solid polyethylene glycols, sodium lauryl sulfate or mixtures thereof. In the case of capsules, tablets and pills, the dosage forms may also comprise buffering agents.

The percentage of active component in the composition and method for treating tumors or cancer can be varied so that a suitable dosage is obtained. The dosage administered to a particular patient is variable depending upon the clinician's judgement using as the criteria: the route of administration, the duration of treatment, the size and condition of the patient, the potency of the active component, and the patient's response thereto. An effective dosage amount of active component can thus readily be determined by the clinician considering all criteria and utilizing his best judgement on the patient's behalf.

The present treatment methods may further comprise administering further cancer treatment methods including, but not limited to, radiation, hormonal, biological and immunotherapy. Radiation, hormonal, biological and immunotherapy may be administered before, after or during the administration of Lucanthone and at least one anti-metabolite.

Radiation may be administered in a variety of fashions. For example, radiation may be electromagnetic or particulate in nature. Electromagnetic radiation useful in the practice of this invention includes, but is not limited, to x-rays and gamma rays. In a preferable embodiment, supervoltage x-rays (x-rays>=4 MeV) may be used in the practice of this invention. Particulate radiation useful in the practice of this invention includes, but is not limited to, electron beams, protons beams, neutron beams, alpha particles, and negative pi mesons. The radiation may be delivered using conventional radiological treatment apparatus and methods, and by intraoperative and stereotactic methods. Additional discussion regarding radiation treatments suitable for use in the practice of this invention may be found throughout Steven A. Leibel et al., Textbook of Radiation Oncology (1998) (publ. W. B. Saunders Company), and particularly in Chapters 13 and 14. Radiation may also be delivered by other methods such as targeted delivery, for example by radioactive “seeds,” or by systemic delivery of targeted radioactive conjugates. J. Padawer et al., Combined Treatment with Radioestradiol Lucanthone in Mouse C3HBA Mammary Adenocarcinoma and with Estradiol Lucanthone in an Estrogen Bioassay, Int. J. Radiat. Oncol. Biol. Phys. 7:347-357 (1981). Other radiation delivery methods may be used in the practice of this invention.

The amount of radiation delivered to the desired treatment volume may be variable. In a preferable embodiment, radiation may be administered in amount effective to cause the arrest or regression of the cancer of a central nervous system in a host, when the radiation is administered with Lucanthone and Pemetrexed. In another embodiment, radiation is administered in at least about 1 Gray (Gy) fractions at least once every other day to a treatment volume, and more preferably radiation is administered in at least about 2 Gray (Gy) fractions at least once per day to a treatment volume, even more preferably radiation is administered in at least about 2 Gray (Gy) fractions at least once per day to a treatment volume for five consecutive days per week. In another embodiment, radiation is administered in 3 Gy fractions every other day, three times per week to a treatment volume. In another embodiment, the first 23 fractions are administered to an initial treatment volume, while another 7 treatment fractions are delivered to a boost treatment volume. In yet another embodiment, a total of at least about 20 Gy, still more preferably at least about 30 Gy, most preferably at least about 60 Gy of radiation is administered to a host in need thereof. In another more preferable embodiment, radiation is administered to the whole brain, rather than to a treatment volume. When irradiating the whole brain, a maximum dosage of 30 Gy is recommended. In a most preferable embodiment, radiation is administered to the whole brain of a host, wherein the host is being treated for metastatic cancer.

In a preferable embodiment, the treatment volume comprises a contrast-enhancing lesion on a CT or MRI scan, more preferably a contrast-enhancing lesion and surrounding edema, still more preferably a contrast-enhancing lesion and surrounding edema on a CT or MRI scan plus at least about a 1 cm margin.

Treatment plans may include, but are not limited to, opposed lateral fields, a wedge pair of fields, rotation or multiple field techniques. CT-guided treatment planning is suggested to improve accuracy in the selection of field arrangements. Isodose distributions for the initial treatment volume and the cone-down treatment volume are suggested for all patients, including those with parallel opposed fields. Composite plans showing dose distribution to the initial treatment volume and the boost treatment volume are desirable. The minimum and maximum dose to the treatment volume are preferably kept to within about 10% of the dose at the center of the treatment volume.

Hormonal therapy involves the manipulation of the endocrine system through exogenous administration of specific hormones, particularly steroid hormones, or drugs which inhibit the production or activity of such hormones (hormone antagonists). Because steroid hormones are powerful drivers of gene expression in certain cancer cells, changing the levels or activity of certain hormones can cause certain cancers to cease growing, or even undergo cell death. Surgical removal of endocrine organs, such as orchiectomy and oophorectomy can also be employed as a form of hormonal therapy.

Immunotherapies are treatments that use natural body substances or drugs made from natural body substances. They stimulate the body to attack cancer cells and overcome side effects caused by other cancer treatments. Immunotherapies use the immune system to reject cancer. The main premise is stimulating the patient's immune system to attack the malignant tumor cells that are responsible for the disease. This can be either through immunization of the patient, in which case the patient's own immune system is trained to recognize tumor cells as targets to be destroyed, or through the administration of therapeutic antibodies as drugs, in which case the patient's immune system is recruited to destroy tumor cells by the therapeutic antibodies.

It will be apparent to those skilled in the art that various modifications and variations can be made in the methods and to the pharmaceutical compositions and kits of the present disclosure without departing from the spirit or scope of the invention. Thus, it is intended that the present disclosure cover the modifications and variations of this invention provided they come within the scope of the appended claims and their equivalents. Additionally, the following examples are appended for the purpose of illustrating the claimed invention, and should not be construed so as to limit the scope of the claimed invention.

### EXAMPLES

### Example 1

Methods: Groups of CD-1 nude mice were xenografted subcutaneously with human H460 non-small cell lung carcinoma cells. Treatments were initiated when tumor growth reached approximately 130 mm³. Treatments were administered 7 times at frequency of once every 4 days and included vehicles, Pemetrexed given intraperitoneally at 200 mg/kg, Lucanthone given orally at 80 mg/kg, and the combinations of Pemetrexed and Lucanthone at the same doses. The tumor growths and body weight change were followed for 28 days.

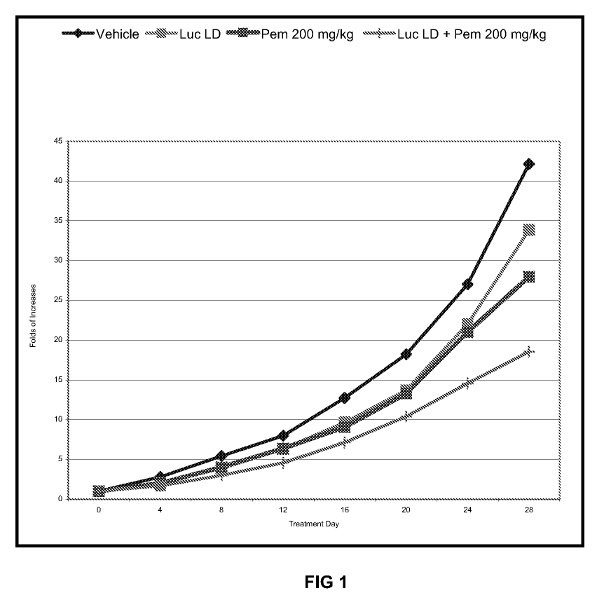

Results: Both Pemetrexed and Lucanthone inhibited tumor growth relative to the vehicles during the 28 days of treatment. For each treatment, fold of increases of the tumor volume were 42, 28, 34 and 19 for vehicles, Pemetrexed, Lucanthone, and the combination of Pemetrexed and Lucanthone, respectively. Compared to the vehicle-treated group, these represent 19%, 33%, and 55% of growth inhibition for groups treated with Lucanthone, Pemetrexed, and the combination of Pemetrexed and Lucanthone, respectively (FIG. 1). During the 28-day period, the body weight changes excluding the tumor weights were −2.9%, −0.9%, −6.1% and −1.7% for vehicles, Pemetrexed, Lucanthone, and the combination of Pemetrexed and Lucanthone, respectively. These body weight data indicate that the treatments were well tolerated with minimum or no general toxicity.

### Example 2

The methods according to Example 1 above may be used for twice daily administrations, once in the morning and once in the evening. The procedure is summarized in Table 1 below.

TABLE 1
Twice daily administration of Lucanthone and Pemetrexed

| Group | Test Material, Dosage | Frequency |
|---|---|---|
| 1 | Vehicle (oral) + Vehicle | Oral: Twice |
| | (intraperitoneal) | daily, once in |
| 2 | Lucanthone (oral), 40 mg/kg/ | the morning and |
| | treatment (80 mg/kg/day and | once in the |
| | 160 mg/kg per 4-day cycle) | evening, given |
| 3 | Pemetrexed (intraperitoneal), 200 mg/kg/ | 8 to 10 hours |
| | treatment/4-day cycle | apart for 2 |
| 4 | Lucanthone (oral), 40 mg/kg/ | consecutive |
| | treatment (80 mg/kg/day and | days in each 4- |
| | 160 mg/kg per 4-day cycle) + | day cycle. |
| | Pemetrexed (intraperitoneal), 200 mg/kg/ | Intraperitoneal: |
| | treatment/4-day cycle | Once in each 4- |
| 5 | Lucanthone (oral), 80 mg/kg/day | day cycle. |
| | and 160 mg/kg per 4-day cycle) | |
| 6 | Lucanthone (oral), 80 mg/kg/day | |
| | and 160 mg/kg per 4-day cycle) + | |
| | Pemetrexed (intraperitoneal), 200 mg/kg/ | |
| | treatment/4-day cycle. | |
| 7 | Pemetrexed (intraperitoneal), 100 mg/kg/ | |
| | treatment/4-day cycle | |
| 8 | Lucanthone (oral), 80 mg/kg/ | |
| | treatment (80 mg/kg/day and | |
| | 160 mg/kg per 4-day cycle) + | |
| | Pemetrexed (intraperitoneal), 100 mg/kg/ | |
| | treatment/4-day cycle | |

Treatment can be initiated approximately 10 days post-tumor cell implantation when the average tumor size is approximately 100 mm³, and once every four days thereafter. The dosage volume can be adjusted once every four days.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. A method of treating cancer in a mammal comprising:
administering Lucanthone to the mammal; and
administering at least one anti-metabolite to the mammal,
wherein said Lucanthone and said at least one anti-metabolite are administered in therapeutically effective amounts.

2. The method of claim 1, wherein said at least one anti-metabolite is folate anti-metabolite.

3. The method of claim 1, wherein said at least one anti-metabolite is Pemetrexed.

4. The method of claim 1, wherein said Lucanthone and said at least one anti-metabolite are administered concurrently.

5. The method of claim 1, wherein said Lucanthone and said at least one anti-metabolite are administered sequentially.

6. The method of claim 5, wherein said Lucanthone and said at least one anti-metabolite are administered within about three hours of each other.

7. The method of claim 5, wherein said Lucanthone and said at least one anti-metabolite are administered within about two hours of each other.

8. The method of claim 5, wherein said Lucanthone and said at least one anti-metabolite are administered within about one hour of each other.

9. The method of claim 1, wherein said cancer is lung cancer.

10. The method of claim 9, wherein said lung cancer is non-small lung carcinoma.

11. The method of claim 1, wherein said therapeutically effective amount of Lucanthone said therapeutically effective amount of at least one anti-metabolite are administered in a single daily dose or divided into more than one daily dose.

12. The method of claim 11, wherein said more than one daily dose is two daily doses.

13. The method of claim 1, wherein said Lucanthone and said at least one anti-metabolite are administered orally.

14. The method of claim 1, wherein said Lucanthone and said at least one anti-metabolite are administered parenterally.

15. The method of claim 1, wherein said Lucanthone and said at least one anti-metabolite are administered in the form of a capsule or tablet.

16. The method of claim 1, wherein said Lucanthone and said at least one anti-metabolite are administered for one or more cycles.

17. The method of claim 16, wherein said one cycle comprises 7 times every 4 days.

18. The method of claim 1, further comprising administering radiation therapy, hormonal therapy or immunotherapy.

19. A pharmaceutical composition comprising therapeutically effective amounts of Lucanthone and at least one anti-metabolite, and a pharmaceutically acceptable carrier.

20. A pharmaceutical kit comprising:
a pharmaceutical composition comprising a therapeutically effective amount of Lucanthone and a pharmaceutically acceptable carrier, and
a pharmaceutical composition comprising a therapeutically effective amount of at least one anti-metabolite and a pharmaceutically acceptable carrier.
